(19) United States
(12) Patent Application Publication
(10) Pub. No.: US 2024/0226426 A1
(43) Pub. Date: Jul. 11, 2024

(54) DELIVERY DEVICE WITH LEAD SCREW INFUSION PUMP

(71) Applicant: Becton, Dickinson and Company, Franklin Lakes, NJ (US)

(72) Inventor: Nicholas Paul ANDERSON, Whitman, MA (US)

(73) Assignee: Becton, Dickinson and Company, Franklin Lakes, NJ (US)

(21) Appl. No.: 18/563,188

(22) PCT Filed: May 26, 2022
(86) PCT No.: PCT/US2022/031001

§ 371 Date: Nov. 21, 2023

Related U.S. Application Data
(60) Provisional application No. 63/193,506, filed on May 26, 2021.

(51) Int. Cl. A61M 5/142

(52) U.S. Cl. CPC A61M 5/14248; A61M 2202/0486

(57) ABSTRACT

A delivery device (10) for delivering medicament, such as insulin, to a patient includes a barrel (20), a delivery mechanism (12), and a pump assembly (14). The pump assembly includes a plunger (54), a drive member (56) and a drive screw (58). The plunger is able to slide within a barrel of the device independently of the drive plate. In one embodiment, the plunger is able to slide axially on the drive screw. The medication, such as insulin, can be introduced to the barrel (20) to fill the device and force the plunger to move axially in the barrel into engagement of the drive plate. The drive plate has a threaded aperture that mates with the drive screw. The drive plate engages the plunger by rotation of the drive screw to dispense the medication.

This application is a U.S. national stage application of International Patent Application No. PCT/US2022/031001, filed May 26, 2022, which claims priority to U.S. Provisional Patent Application No. 63/193,506, filed May 26, 2021, which is hereby incorporated by reference in its entirety.

### FIELD OF THE INVENTION

The present invention relates to medical devices, and more particularly, to a medication dispensing and/or delivery device with at least one fluid channel to direct the medicament, such as insulin, to a delivery mechanism for delivering the medicament to a patient. The delivery device includes a pump mechanism with a manually movable plunger and screw operated drive assembly.

### BACKGROUND OF THE INVENTION

Diabetes is a group of diseases characterized by high levels of blood glucose resulting from the inability of diabetic patients to maintain proper levels of insulin production when required. Diabetes can be dangerous to the affected patient if it is not treated, and it can lead to serious health complications and premature death. However, such complications can be minimized by utilizing one or more treatment options to help control the diabetes and reduce the risk of complications.

The treatment options for diabetic patients include specialized diets, oral medications and/or insulin therapy. The main goal of diabetes treatment is to control the diabetic patient's blood glucose or sugar level. However, maintaining proper diabetes management may be complicated because it has to be balanced with the activities of the diabetic patient. Type 1 diabetes (T1D) patients are required to take insulin (e.g., via injections or infusion) to move glucose from the bloodstream because their bodies generally cannot produce insulin. Type 2 diabetes (T2D) patients generally can produce insulin but their bodies cannot use the insulin properly to maintain blood glucose levels within medically acceptable ranges. In contrast to people with T1D, the majority of those with T2D usually do not require daily doses of insulin to survive. Many people are able to manage their condition through a healthy diet and increased physical activity or oral medication. However, if they are unable to regulate their blood glucose levels, they will be prescribed insulin. For example, there are an estimated 6.2 million Type 2 diabetes patients (e.g., in the United States, Western Europe and Canada) taking multiple-daily-injections (MDI) which consist of a 24-hour basal insulin and a short acting rapid insulin that is taken at mealtimes for glycemic management control.

For the treatment of Type 1 diabetes (T1D) and sometimes Type 2 diabetes (T2D), there are two principal methods of daily insulin therapy. In the first method, diabetic patients use syringes or insulin pens to self-inject insulin when needed. This method requires a needle stick for each injection, and the diabetic patient may require three to four injections daily. The syringes and insulin pens that are used to inject insulin are relatively simple to use and cost effective.

An effective method for insulin therapy and managing diabetes is infusion therapy or infusion pump therapy in which an insulin pump is used. The insulin pump is able to provide continuous infusion of insulin to a diabetic patient at varying rates to more closely match the functions and behavior of a properly operating pancreas of a non-diabetic person that produces the required insulin, and the insulin pump can help the diabetic patient maintain his/her blood glucose level within target ranges based on the diabetic patient's individual needs. Infusion pump therapy requires an infusion cannula, typically in the form of an infusion needle or a flexible catheter, that pierces the diabetic patient's skin and through which infusion of insulin takes place. Infusion pump therapy offers the advantages of continuous infusion of insulin, precision dosing, and programmable delivery schedules.

Insulin pumps advantageously deliver insulin over time rather than in single injections, typically resulting in less variation within the blood glucose range that is recommended. In addition, insulin pumps may reduce the number of needle sticks which the diabetic patient must endure, and improve diabetes management to enhance the diabetic patient's quality of life. For example, many of the patients who are prescribed insulin therapy can be expected to convert from injections to infusion therapy due to an unmet clinical need for improved control. That is, a significant number of the patients who take multiple-daily-injections are not achieving target glucose control or not adhering sufficiently to their prescribed insulin therapy.

To facilitate infusion therapy, there are generally two types of insulin pumps, namely, conventional pumps and patch pumps. Conventional pumps use a disposable component, typically referred to as an infusion set, tubing set or pump set, which conveys the insulin from a reservoir within the pump into the skin of the user. The infusion set includes a pump connector, a length of tubing, and a hub or base from which a cannula, in the form of a hollow metal infusion needle or flexible plastic catheter, extends. The base typically has an adhesive that retains the base on the skin surface during use. The cannula can be inserted onto the skin manually or with the aid of a manual or automatic insertion device. The insertion device may be a separate unit employed by the user.

Another type of insulin pump is a patch pump. Unlike a conventional infusion pump and infusion set combination, a patch pump is an integrated device that combines most or all of the fluidic components in a single housing. Generally, the housing is adhesively attached to an infusion site on the patient's skin, and does not require the use of a separate infusion or tubing set. A patch pump containing insulin adheres to the skin and delivers the insulin over a period of time via an integrated subcutaneous cannula. Some patch pumps may wirelessly communicate with a separate controller device (as in one device sold by Insulet Corporation under the brand name OmniPod®), while others are completely self-contained. Such patch pumps are replaced on a frequent basis, such as every three days, or when the insulin reservoir is exhausted. Otherwise, complications may occur, such as restriction in the cannula or the infusion site.

As patch pumps are designed to be a self-contained unit that is worn by the patient, preferably, the patch pump is small, so that it does not interfere with the activities of the user. Thus, to minimize discomfort to the user, it would be preferable to minimize the overall thickness of the patch pump. However, to minimize the thickness of the patch pump, the size of its constituent parts should be reduced as much as possible.

In current patch pump designs, tubes, such as plastic tubes, are employed as fluid pathways to route fluid flow from one internal component to another. For example, a tube can connect a medicament reservoir with a delivery needle, but the space required to internally house such a tube adds to the overall size of the patch pump. The use of tubes can increase cost and can result in additional complexity during automated device assembly processes. For example, such device assembly includes connecting the tubes, which adds steps to the assembly process. In addition, preventing leaks from such connections can give rise to additional challenges.

Accordingly, a need exists for an improved pump construction and mechanism for controlling the volume of medication in the delivery device.

### SUMMARY

A feature of the present dispensing and delivery device, such as a patch pump or infusion pump, is dispensing device including a pump assembly connected to a delivery mechanism of the device. The delivery mechanism can be a needle, cannula or flexible catheter of a patch pump or infusion set. The pump mechanism is configured to be filled manually by the user to contain a predetermined volume of the insulin or other medicament for dispensing from the device and delivery to the user.

The pump assembly in one embodiment has a barrel, a plunger, a drive assembly having a drive member for engaging the plunger, and an actuator, such as a drive screw coupled to the drive member. The drive screw extents through the plunger where the plunger is able to slide axially on the drive screw. In an initial position, the plunger is oriented at one end of the barrel on the drive screw. The medication, such as insulin, is introduced into the barrel to create a positive pressure to force the plunger to move or slide toward the drive member. The drive member is oriented at a position to define a predetermined volume in the barrel so that when the plunger contacts the drive member, the predetermined volume of medication is provided in the barrel. Rotation of the drive screw moves the plunger to dispense the medication.

The pump assembly can be configured to accommodate variable fill volumes depending on the intended needs. The pump mechanism can be filled with a selected volume determined by the position of the drive mechanism as determined by a controller or user.

In one embodiment, the delivery device includes a barrel, a plunger, a drive assembly including a drive member and a drive screw, and a controller. The controller is able to position the drive plate in a predetermined position within the barrel corresponding to a predetermined volume of the medication to be filled into the barrel. The medication, such as insulin, is introduced into the barrel to slide the plunger over the drive screw from an end of the barrel toward the drive plate. The controller can include a sensor to detect whether the intended volume of the medication is filled into the barrel. The drive assembly is activated to prime the delivery device, where the sensor is able to determine the location of the plunger in the barrel after medication is introduced to indicate the volume of the medication in the barrel. In one embodiment, the sensor measures the time between activation and contact of the drive member with the plunger or the distance travelled by the drive member to contact the drive member to indicate the volume of medication in the barrel. The sensor can provide a prompt to the user corresponding to the volume of the medication in the barrel. The medication is then dispensed by rotation of the drive screw and the axial movement of the drive member and plunger.

The delivery device includes a medication reservoir, a delivery mechanism for delivering the medication to the patient, and a pump mechanism for delivering the medication from the reservoir to the delivery mechanism. The delivery mechanism is typically a needle, cannula or catheter positioned or inserted in the patient for intradermal delivery.

The pump mechanism in one embodiment includes a plunger having a central opening extending axially through the plunger. A resilient and/or flexible seal member is positioned in the central opening where the seal has a central opening for engaging the outer surface of a drive screw. The seal member forms a fluid seal between the seal and the drive screw. The plunger is able to slide axially on the drive screw during filling of the delivery device and maintain a seal during delivery of the insulin.

The pump mechanism in one embodiment includes a plunger with an engagement mechanism for connecting with a drive screw. The engagement mechanism can be tabs on the plunger that engage a respective connector on the drive plate. In other embodiments, the drive plate can have one or more projecting pins that align with respective apertures in the plunger when the plunger mates with the drive plate.

The delivery device in one embodiment is configured for delivering a medicament to a patient via a needle, cannula or flexible catheter positioned in the patient. The device has a housing, which includes a reservoir for containing the medicament. A delivery cannula delivers the medicament into the skin of the patient. In one embodiment, a base has a bottom surface for orienting toward the skin of the patient and a top surface facing the internal space.

The delivery device can have a housing and a base enclosing the housing to form an internal cavity for receiving the pump mechanism and other operating components for the delivery device. The delivery device is particularly suitable for delivering insulin at a controlled rate through a cannula or catheter to the patient. The delivery device is shown as a patch pump or infusion pump although the delivery device can have other forms, such as pump mechanism for use with an infusion set. At least one surface of the housing or the base has an integrally formed fluid channel to deliver the insulin from one component of the delivery device to the cannula or catheter

The features of the invention in one embodiment are provided by a medication delivery device comprising a barrel having an internal cavity configured for receiving a medication; a plunger positioned in an internal cavity and being movable between a first end and a second end of said barrel; and a drive assembly including an actuator and drive member positioned in said internal cavity between a first end and second end of said barrel, said plunger being movable independently of said drive member, said drive member coupled to said actuator for moving said drive member into contact with said plunger and moving said plunger toward said first end of said cavity to dispense the medication.

In another embodiment, the features are provided by a medication delivery device comprising a barrel having an internal cavity for receiving a medication, said internal cavity having an outlet connected to a delivery assembly for introducing the medication to a patient, said internal cavity having an inlet for introducing the medication into said internal cavity, a movable plunger in said internal cavity, said plunger being movable from a first end toward a second end of said barrel by introducing the medication into said internal cavity; and a drive assembly including a drive screw and a drive member having internal threads for coupling to said drive screw for moving axially with respect to an axis of said drive screw, said plunger being movable toward said second end of said barrel into contact with said drive member, and where rotation of said drive screw moves said drive member and plunger toward said first end to dispense the medication through said outlet of said barrel to said delivery assembly.

The features of the delivery device are further provided by a medication delivery device comprising a barrel having an internal cavity for receiving a medication, said internal cavity having an outlet connected to a delivery assembly for introducing the medication to a patient, said internal cavity having an inlet for introducing the medication into said internal cavity, a movable plunger in said internal cavity between said first end and second end; a drive assembly having a drive screw and actuator coupled to said drive screw for moving axially on said drive screw, said drive screw extending axially through said actuator and said plunger, and said plunger being movable axially on said drive screw independent of rotation of said drive screw; and a drive motor coupled to said drive screw for rotation of said drive screw.

Additional and/or other aspects and advantages of the present delivery device is set forth in the description that follows or will be apparent from the description, or may be apparent by practice of the invention. The present invention may comprise delivery devices and methods for forming and operating same having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The various aspects and advantages of embodiments of the delivery device will be more readily appreciated from the following detailed description, taken in conjunction with the accompanying drawings, of which:

FIG. 1 is a perspective view of a delivery device constructed in accordance with an illustrative embodiment;

FIG. 2 is perspective view of the delivery device of FIG. 1 showing the cannula extending from the bottom face of the delivery device;

FIG. 3 is an exploded view of the various components of the delivery device of FIG. 1;

FIG. 4 is a side view in cross section of the delivery device;

FIG. 5 is a schematic illustration of the pump mechanism, motor and control device;

FIG. 6 is a perspective view of the pump mechanism of the delivery device in one embodiment;

FIG. 7 is a side view in cross section of the pump mechanism of FIG. 6;

FIG. 8 is a rear perspective view of the pump mechanism;

FIG. 9 is an explode view of the pump mechanism;

FIG. 10 is perspective view of the plunger in one embodiment;

FIG. 11 is a cross sectional view of the drive plate of the pump mechanism;

FIG. 12 is a partial cross sectional side view of the pump mechanism showing the plunger in the position for filling the barrel;

FIG. 13 is partial cross sectional side view of the pump mechanism showing the plunger in the filled position;

FIG. 14 is a partial cross sectional side view of the pump mechanism showing the plunger dispensing the fluid;

FIG. 15 is cross sectional side view of the pump mechanism showing the connection between the plunger and the drive plate;

FIG. 16 is a cross sectional side view of the pump mechanism showing the latching connection between the plunger and drive plate;

FIG. 17 is a cross sectional view of the pump mechanism of FIG. 12;

FIG. 18 is a cross sectional view of the pump mechanism of FIG. 13;

FIG. 19 is a cross sectional view of the pump mechanism of FIG. 14;

FIG. 20 is cross sectional view of the pump mechanism is an alternative mode of operation; and

FIG. 21 is cross sectional view of the pump mechanism of FIG. 20 showing the plunger engaged with the drive plate.

### DETAILED DESCRIPTION OF EMBODIMENTS

Reference will now be made in detail to embodiments of the present invention, which are illustrated in the accompanying drawings, wherein like reference numerals refer to like elements throughout. The embodiments described herein exemplify, but do not limit, the present invention by referring to the drawings.

It will be understood by one skilled in the art that this disclosure is not limited in its application to the details of construction and the arrangement of components set forth in the following description or illustrated in the drawings. The embodiments herein are capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting. Any of the embodiments and/or elements and features disclosed herein may be combined with one another to form various additional embodiments not specifically disclosed, as long as they do not contradict or are not inconsistent with each other. Terms of degree, such as “substantially”, “about” and “approximately” are understood by those skilled in the art to refer to reasonable ranges around and including the given value and ranges outside the given value, for example, general tolerances associated with manufacturing, assembly, and use of the embodiments. The term “substantially” when referring to a structure or characteristic includes the characteristic that is mostly or entirely.

The illustrative embodiments are described with reference to diabetes management using insulin therapy. It is to be understood that these illustrative embodiments can be used with different drug therapies and regimens to treat physiological conditions other than diabetes using different medicaments other than insulin.

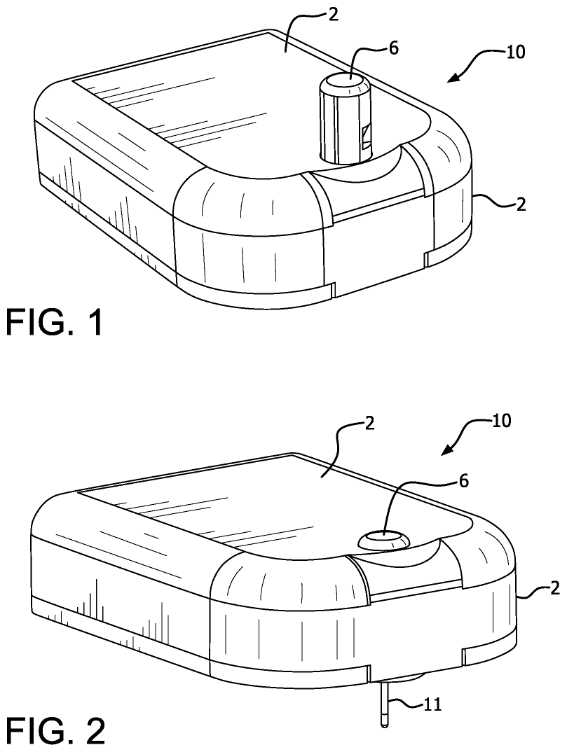

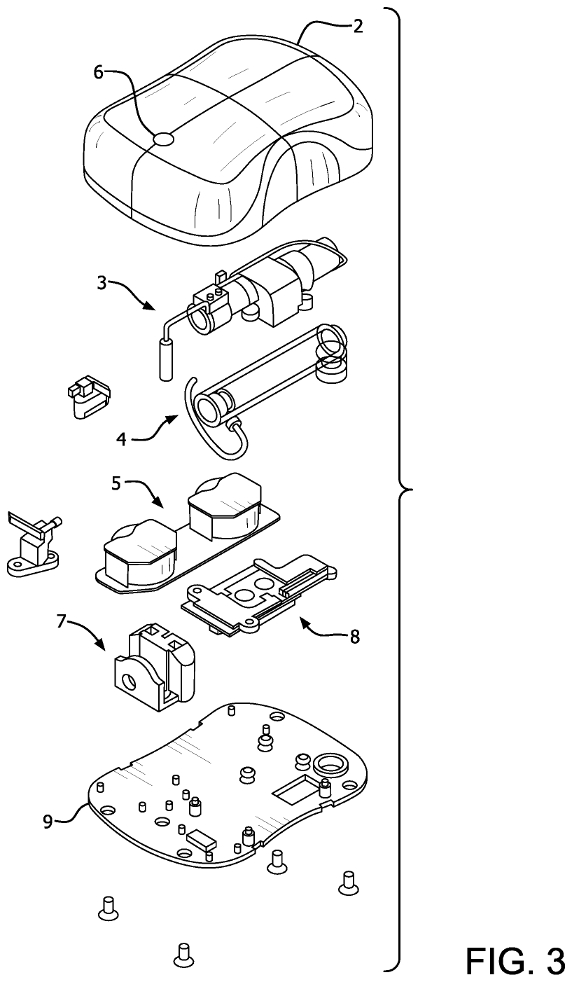

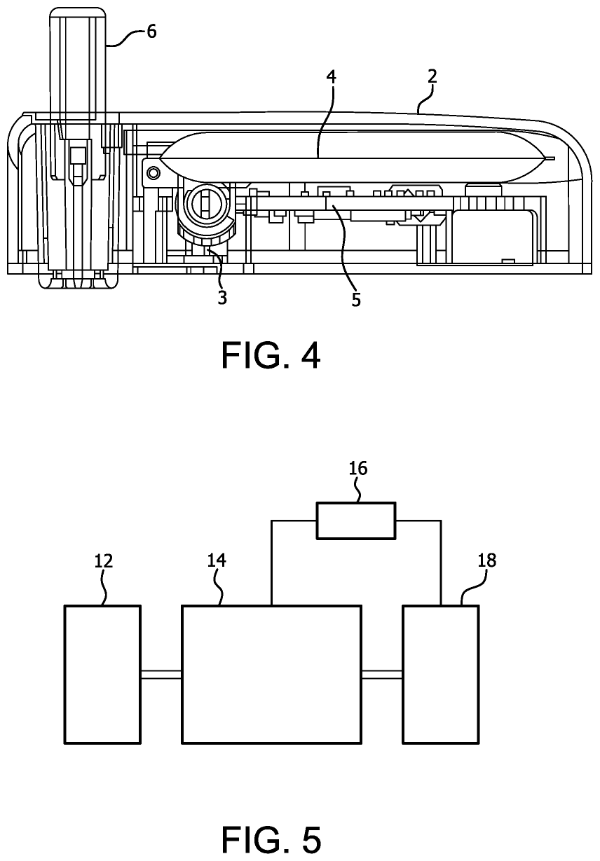

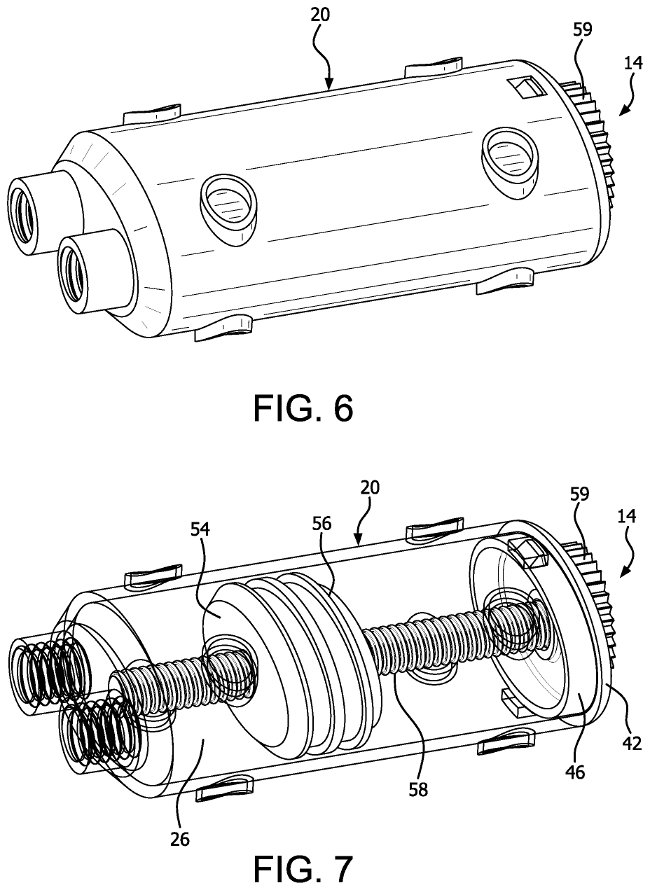

FIG. 1 and FIG. 2 are perspective views of an exemplary embodiment of a medicament delivery device shown as a patch pump 10. In other embodiments, the delivery device can have other structures and delivery mechanism, such as an infusion set and pump arrangement. The patch pump as described herein is configured for delivering insulin in the form of an insulin solution to a patient at a controlled rate. FIG. 3 is an exploded view of the various components of the patch pump of FIG. 1, illustrated with a housing forming a main cover.

The various components of the patch pump 10 include a reservoir 4 for storing insulin; a pump 3 for pumping insulin out of the reservoir 4; a power source 5 in the form of one or more batteries; a delivery and insertion mechanism 7 for inserting an inserter needle with a cannula into a user's skin; control electronics 8 in the form of a circuit board with optional communications capabilities to outside devices such as a remote controller and computer, including a smart phone; a pair of dose buttons on the housing 2 for actuating an insulin dose, including a bolus dose. The insertion mechanism 7 is actuated by manually depressing the button 6 on the cover to deploy the insertion needle and cannula 11 as shown in FIG. 2. A base 9 for supporting the components may be attached to the housing 2 by suitable fasteners or snap connectors. The patch pump 10 also includes various fluid connector lines that transfer insulin pumped out of the reservoir 4 to the infusion site. The cannula of the delivery mechanism can be a rigid cannula or flexible catheter as known in the art.

The wearable medical delivery device (e.g., insulin delivery device (IDD) such as patch pump 10 can be operable in conjunction with a remote controller that communicates wirelessly with the pump 10 that can include a graphical user interface (GUI) display for providing a user visual information about the operation of the patch pump 10 such as, for example, configuration settings, an indication when a wireless connection to the patch pump is successful, and a visual indication when a dose is being delivered, among other display operations. The display can include a touchscreen display that is programmed to allow a user to provide touch inputs such as a swipe to unlock, swipe to confirm a request to deliver a bolus, and selection of confirmation or settings buttons, among other user interface operations.

FIG. 5 is a schematic illustration of the delivery device 10 according an illustrated embodiment including the delivery mechanism 12, such as the cannula or other infusion device as known in the art for delivering the medication to the patient. The delivery mechanism 12 is connected to the pump assembly 14 for receiving the medication from the pump assembly 14. A drive motor 16 is shown connected to the pump assembly 14. A controller 18 is connected to the pump assembly 14 and the drive motor 16 as discussed in further detail below.

In the embodiment shown, the pump assembly 14 is configured for delivering a controlled amount of the medication to the delivery mechanism for delivering the mediation to the patient in a controlled manner. The delivery device is particularly suitable for delivering insulin to a patient from a patch pump or infusion set.

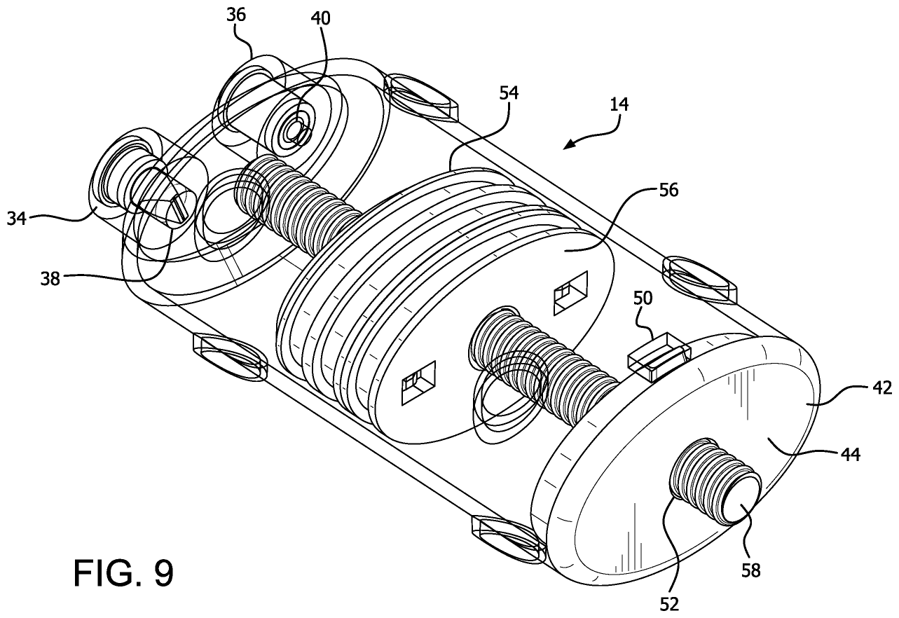

The pump assembly 14 as shown in the drawings include a barrel 20 configured for mounting to the base of the delivery device 10. The barrel 20 in the embodiment shown, has a substantially oval cross sectional shape with an outer wall 22 having an inner wall surface 24 forming an interior cavity 26. The barrel 20 has first end 28 and an open second end 30. The first end 20 has an end wall 32 with an inlet port 34 defined by a collar extending from the end wall for receiving the medication and an outlet port 36 formed by a collar extending from the end wall for discharging the medication to the delivery mechanism 12. As shown in FIG. 9, the inlet port 34 includes a self-closing one-way valve 38 for introducing the medication into the barrel and preventing leakage of the medication. The valve 38 is configured to be opened by inserting a tip or nozzle of a medication container for filling the delivery device. The outlet port 36 includes a self-closing, one-way valve 40 to allow the medication to flow to the delivery mechanism 12 and prevent back flow into the pump assembly. The inlet port 34 is accessible to the user to feed the insulin or other medication through the inlet port and valve 38 by a inserting the tip of a supply container though the valve. The outlet port 36 is connected to the delivery mechanism by a suitable conduit or feed tube, which is not shown in the drawings for convenience of illustration.

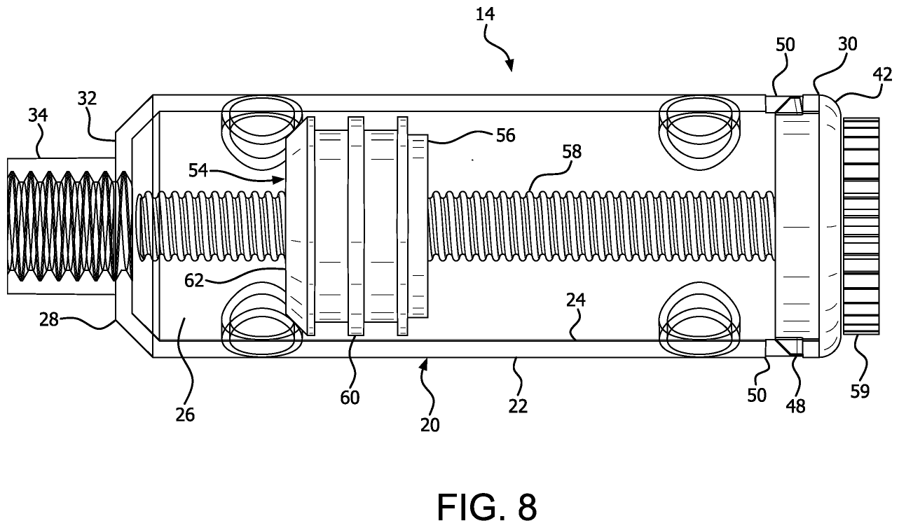

The open end 30 of the barrel 20 is closed by an end cap 42 that is coupled to the barrel. In the embodiment shown, the end cap 42 has a base portion 44 with a shape and dimension corresponding to the outer dimension of the barrel 20 and a collar 46 extending from the base portion 44 with a shape and dimension corresponding to the inner shape and dimension of the barrel 20. In the embodiment shown, the collar 46 has at least one coupling tab 48 for mating with a corresponding aperture 50 in the side wall 22 of the barrel 20. In one embodiment, two coupling tabs 48 and respective apertures 50 are provided to couple the end cap to the barrel 20. As shown in FIGS. 8 and 9 the base portion has a central opening 52.

The pump assembly 14 includes a plunger 54, a drive assembly, and a drive motor 16. The drive assembly includes a drive member, shown as a drive plate 56 and an actuator shown as a drive screw 58. The motor 16 is coupled to the drive screw 58 by a gear 59 or other mechanical connection for rotating the drive screw 58. The drive screw 58 is axially fixed relative to the barrel and is able rotate about the longitudinal axis of the barrel. The plunger 54 in the embodiment shown can move independently of the drive plate 56 and drive screw 58. The plunger 54 has an outer surface with radially extending ribs 60 for contacting and sealing with the inner surface of the barrel 20 and sliding axially relative to the barrel 20. The ribs 60 form a seal with the inner surface of the barrel for dispensing the medication when the plunger 54 is moved toward the first end of the barrel. The plunger 54 has a distal face 62 and a proximal face 64.

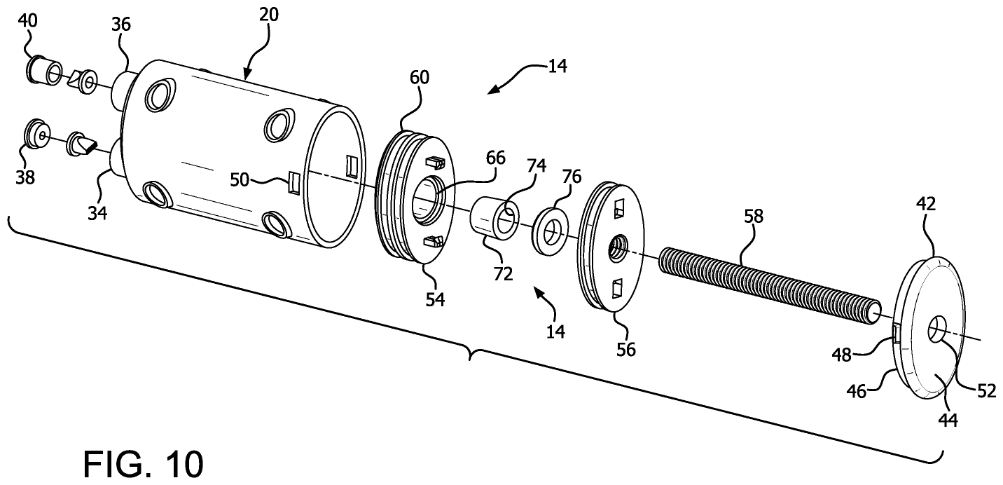

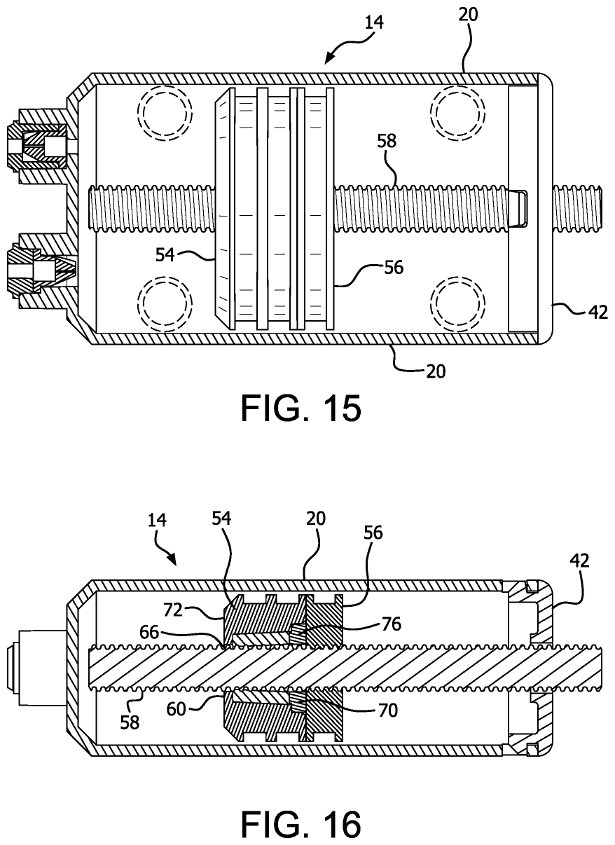

As shown in FIGS. 9 and 10, the plunger 54 has a central opening 66 extending through the plunger 54 between the distal face 62 and the proximal face 64 with a dimension for receiving the drive screw 58. Referring to FIGS. 9 and 10, the central opening 66 has a flange 68 extending radially inward at the distal face 62 forming a shoulder at the distal end of an enlarged portion 70 at the proximal face 64 as shown in FIGS. 15 and 16. A seal in the form of a sleeve 72 is positioned in the enlarged portion 70 as shown in FIGS. 9 and 10. The sleeve 72 has an outer surface to fit in the enlarged portion 70, such as by a friction fit or adhesive and inner surface 74 with a dimension complementing the outer dimension of the drive screw 58. The sleeve 72 can be made of a flexible or resilient plastic or elastomeric polymer that is able to form a fluid resistant or fluid tight seal between the drive plunger 54 and the drive screw 58. In the embodiment shown, a retainer ring 76 fits in the open end of the enlarge portion 70 of the plunger 54 for retaining the sleeve 72 in the plunger 54. The retainer ring 76 has a central opening for receiving the drive screw 58 and is able to slide axially over the drive screw 58 as shown.

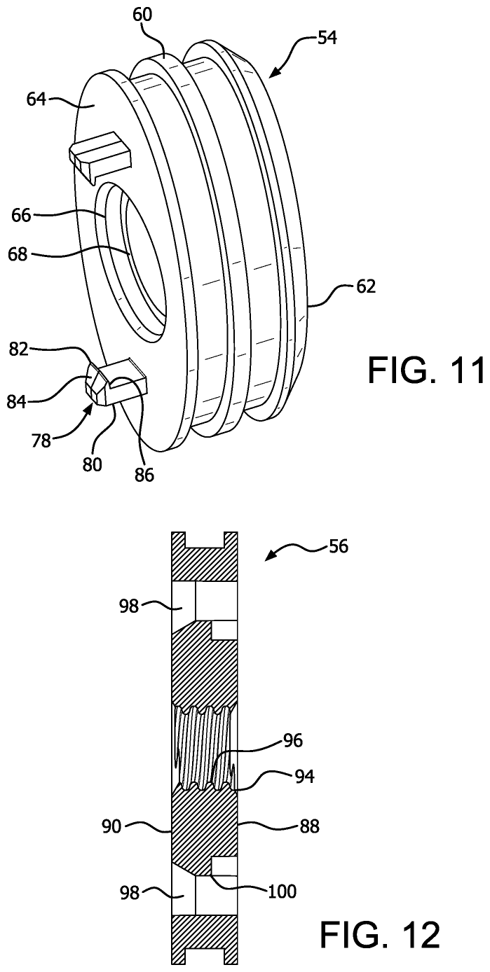

In the embodiment shown in FIG. 11, the proximal face 64 of the plunger 54 includes coupling members shown as coupling tabs 78 for coupling with the drive plate 56. The coupling tabs 78 include an axially ending leg 80 with an inwardly projecting hook 82. The hook 82 has an inclined outer surface 84 for sliding over a respective latch of the drive plate 56 and a substantially flat inner surface 86 for hooking to the drive plate 56.

The drive plate 56 is configured for moving axially in the barrel 20 and engaging the plunger to dispense the contents of the barrel. In the embodiment shown, the drive plate has a shape and dimension received in the cavity of the barrel for axial movement. As shown in FIG. 9, the drive plate 56 has a proximal face 88, a distal face 90, and an outer edge having radially extending ribs 92. The ribs 92 can have a dimension to provide a guide mechanism for enabling the drive plate to slide axially in the barrel. A threaded hole 94 having internal threads 96 extends axially through the drive plate between the distal face and proximal face for receiving the drive screw 58 where rotation of the drive screw 58 causes axial movement of the drive plate relative to the barrel.

In one embodiment as shown in FIG. 11, the drive plate 58 has coupling members for coupling with the respective coupling members of the plunger 54. The coupling members as shown include a through passage 98 extending axially through the drive plate 58. A projection 100 extends inwardly into the passage 98 for latching with the coupling tabs 78 of the plunger 54. The projection 100 has an inclined distal face 102 to assist the coupling tabs 78 in sliding over the projection 100 and a flat proximal face 104 for mating with the hook portion of the coupling tabs 78 of the plunger. Alternative mechanisms can be used to couple the drive plate and plunger, such as a snap fit connector or pins extending from one of the plunger or drive plate that are received in a complementing hole in the other of the plunger or drive plate by a friction fit.

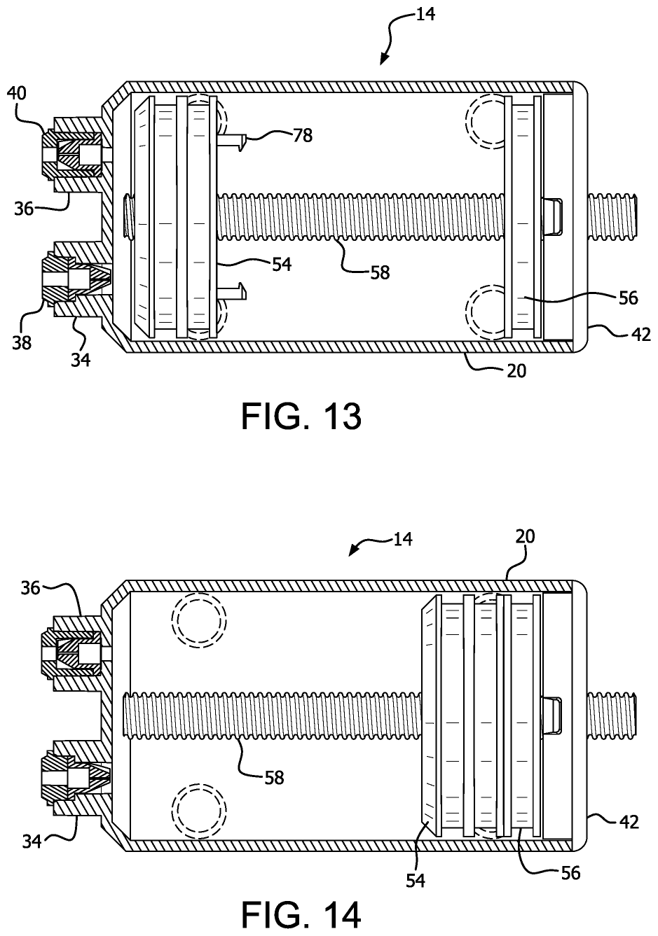

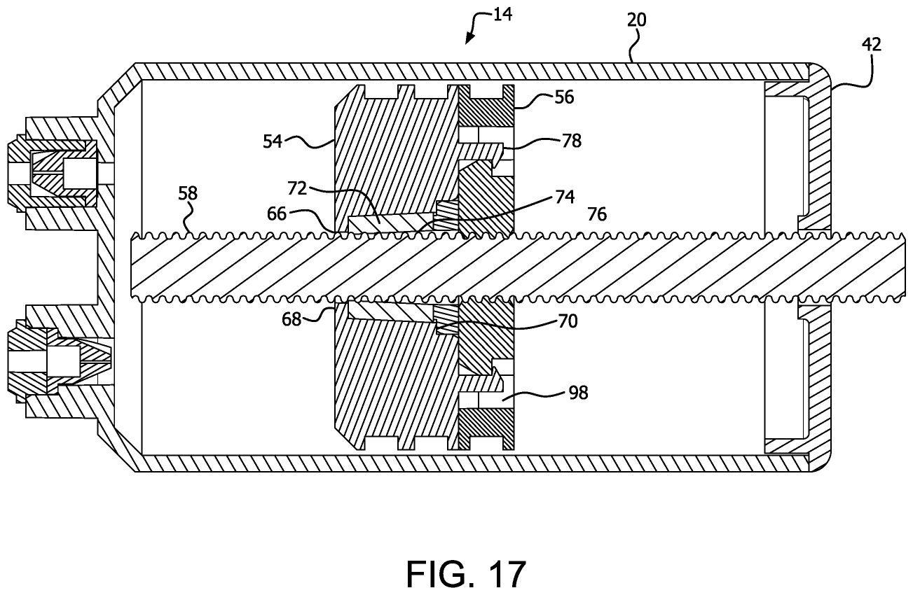

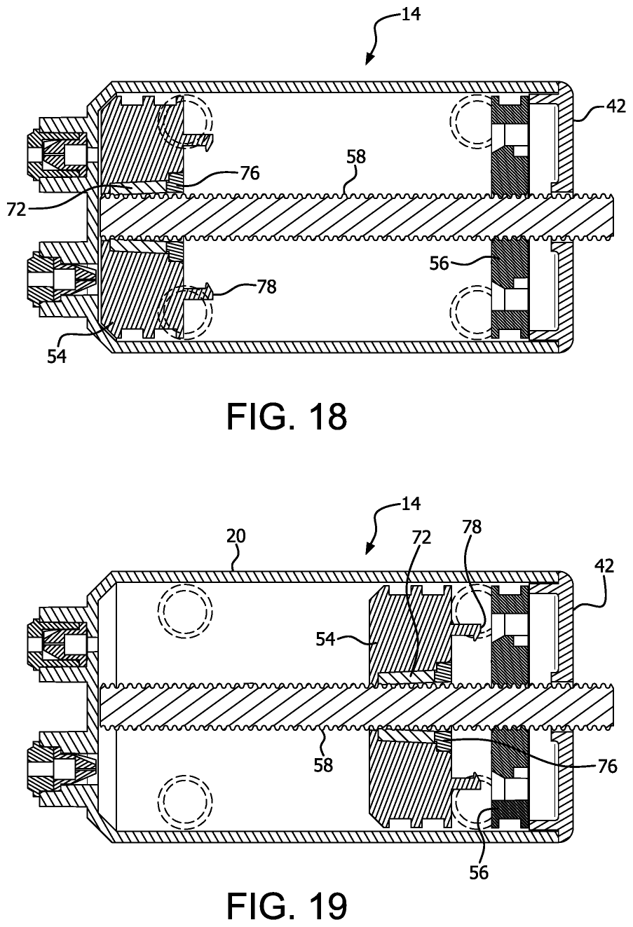

The pump assembly 14 is configured to be filled by the user before use and then actuated by the motor to deliver the insulin to the patient at a controlled dosage. In one embodiment for a single volume manual fill, the pump assembly is initially configured as shown in FIG. 13 and FIG. 18, with the plunger 54 positioned at the distal end of the barrel 20 and the drive plate 56 at the proximal end to provide a maximum capacity of the barrel 20 for the medication, such as insulin. The insulin is introduced by the user through the inlet port 34 by a syringe or other suitable device into the cavity of the barrel 20. The insulin is introduced to generate a positive pressure in the barrel 20 and the distal end of the barrel, which causes the plunger to move toward the drive plate 56 and the proximal end of the barrel as shown in FIG. 14 and FIG. 19. When the plunger engages the drive plate 58, the coupling tabs 78 of the plunger 54 engage the drive plate 56. The drive motor 16 rotates the drive screw 58 at a controlled rate and timing to drive the drive plate and plunger forward as shown in FIGS. 14-17 and 20 to deliver a predetermined dosage of the insulin though the outlet port 36 to the delivery mechanism.

In the embodiment illustrated, a single drive screw is shown for advancing the drive member relative to the barrel. The drive assembly for the drive member can be other mechanism that are able to advance the drive member. In one embodiment, the drive screw engages the drive member but does not extend through the drive member or the plunger. The drive screw can include several concentric, telescoping threaded members that can telescope axially by rotation of one or more of the telescoping members.

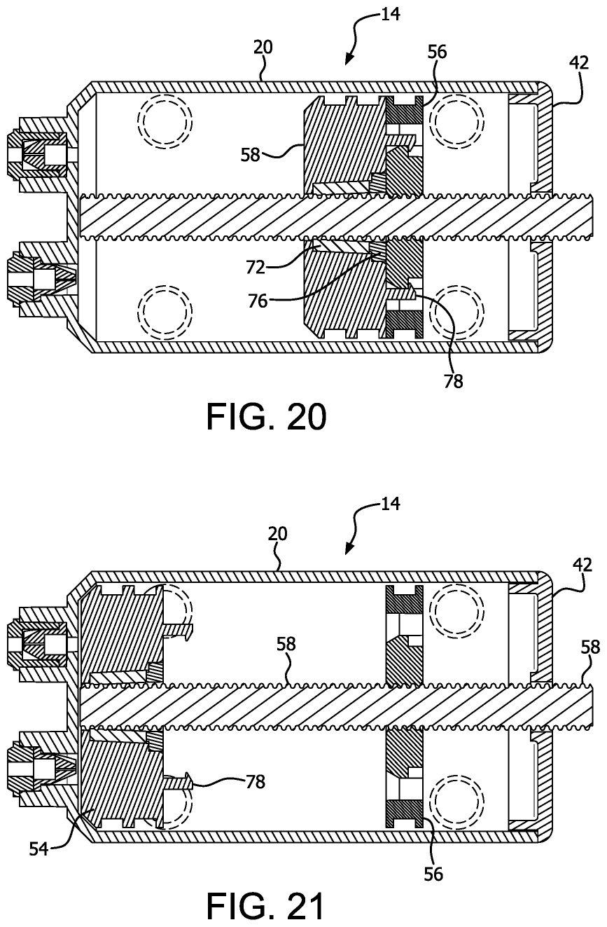

In another embodiment, the controller 18 is activated to select a predetermined volume of insulin that is less than the full capacity of the barrel 20. The controller 18 actuates the drive motor 16 to advance the drive plate 56 to a position corresponding to the intended volume in the distal end of the barrel as shown in FIG. 20. The controller is able to provide different positions of the drive plate and different volumes depending on the particular need for the patient. The plunger 54 is positioned toward the inlet port of the barrel as shown in FIG. 20. The insulin is then introduced through the inlet port 34 into the cavity of the barrel 20 to move the plunger 54 into contact with the drive plate 56. The controller 18 can then actuated the drive motor to rotate the drive screw and advance the drive plate and the plunger to provide the dosing operation.

The controller 18 is connected to the drive motor 16 to operate the drive screw during the filling and delivery operations. The controller can include a user input and display mechanism to enable the user to operate the delivery device. The user is able to program and set a volume into the controller 18 so that the controller 18 actuates the drive motor to move and position the drive plate at a location within the barrel corresponding to the selected volume The controller can then provide a prompt to the user that the volume has been selected and to direct the user to fill the barrel. The user is then able to fill the barrel to the selected volume where the pressure in the barrel moves the plunger into contact with the drive plate. The controller can be connected to the barrel of the pump mechanism to sense and determine the position of the plunger relative to the drive plate to ensure the correct volume of insulin has been introduced into the barrel. The controller can sense whether the proper volume of insulin has been supplied to the barrel and provide feedback to the user when a correct volume is introduced or when an insufficient volume has been introduced. In the event the user does not fill the barrel with the proper or intended volume, during the priming stage of the device, the controller can monitor the current applied to the drive motor as the drive plate is advanced toward the plunger. A low current can be detected while the drive plate moves toward the plunger by the low resistance or torque applied by the drive motor. The controller can also detect the time the drive motor operates with low resistance to indicate the distance travelled by the drive plate and the volume of the insulin in the barrel. The controller can maintain the priming cycle until a spike in current is detected thereby indicating the drive plate contacting the plunger and indicating the volume of the medication in the barrel. A large signal or spike in current is produced when the torque increases by the drive plate engaging the plunger to break the static friction between the plunger and the inner surface of the barrel.

During use, the bottom surface of the delivery device is oriented toward the skin of the patient. In some embodiments, the bottom surface includes an adhesive that removably attaches the base to the skin of the patient. Alternatively, an adhesive pad as illustrated in FIG. 1, adheres to both the bottom surface and the skin of the patient. The adhesive is selected to ensure compatibility with human skin to prevent undesired reactions. Also, compatibility of the adhesive and the insulin is considered in case the adhesive and the insulin accidentally mix.

Although only a few embodiments of the present device are shown and described, the present device is not limited to the described embodiments. Instead, it will be appreciated by those skilled in the art that changes may be made to these embodiments without departing from the principles and spirit of the device. Different embodiments can be combined with other embodiments as long as they are not inconsistent with each other. It is particularly noted that those skilled in the art can readily combine the various technical aspects of the various elements of the various exemplary embodiments that have been described above in numerous other ways, all of which are considered to be within the scope of the disclosure and equivalents thereof.

1. A delivery device for a medicament comprising:
a barrel having an internal cavity configured for receiving a medication, said barrel having a first end and a second end;
a plunger positioned in said internal cavity and being movable between said first end and said second end of said barrel; and
a drive assembly including an actuator and drive member positioned in said internal cavity between said first end and second end of said barrel, said drive member being movable independently of said plunger, said drive member coupled to said actuator for moving said drive member into contact with said plunger and moving said plunger toward said first end of said cavity to dispense said medication.

2. The delivery device according to claim 1, wherein said plunger is movable independent of said drive assembly.

3. The delivery device according to claim 1, wherein said plunger is configured to move from said first end of said barrel toward said second end of said barrel by introducing the medication into said barrel at said first end.

4. The delivery device according to claim 3, wherein said plunger is spaced from said drive member when said plunger is at said first end of said barrel and engages said drive member when said plunger moves toward said second end of said barrel.

5. The delivery device according to claim 4, wherein said plunger couples to said drive member when said plunger engages said drive member.

6. The delivery device according to claim 3, wherein said plunger is axially slideable on said actuator.

7. The delivery device according to claim 6, wherein said actuator comprises a threaded screw that is axially fixed relative to said barrel, and said drive member comprises drive plate having a threaded opening receiving said threaded screw.

8. The delivery device according to claim 7, further comprising a controller operatively connected to said drive assembly for positioning said drive member in a predetermined location in said barrel corresponding to a predetermined volume of the medication in said barrel.

9. A medication delivery device for delivering a medicament, said delivery device comprising:
a barrel having an interior cavity configured for receiving the medication, said barrel having a first end and second end;
a plunger in said internal cavity of said barrel and movable between said first end and said second end;
a drive assembly having a drive screw and a drive member coupled to said drive screw for moving axially on said drive screw, said drive screw extending axially through said drive member and said plunger, and said plunger being movable axially on said drive screw independently of rotation of said drive screw; and
a drive motor operatively coupled to said drive screw for rotation of drive screw.

10. The medication delivery device according to claim 9, wherein said plunger is slideable axially on said drive screw.

11. The medication delivery device according to claim 10, wherein said plunger has an opening extending between a proximal face and a distal face for receiving said drive screw.

12. The medication delivery device according to claim 11, wherein said plunger includes a seal in said opening for forming a fluid tight seal between said drive screw and said plunger, and where said seal slides axially over said drive screw.

13. The medication delivery device of claim 9, wherein said first end of said barrel includes an inlet port for filling said barrel with the medication and an outlet port for dispensing said medication.

14. The medication delivery device of claim 13, wherein said plunger is movable from a first position in said barrel to a second position by introducing the medication into said barrel through said inlet port.

15. The medication delivery device of claim 14, wherein said plunger engages said drive member when said plunger moves to said second position.

16. The medication delivery device of claim 9, further comprising a controller operatively coupled to said drive assembly for positioning said drive member in a predetermined location in said barrel corresponding to a predetermined volume of medication in said barrel.

17. A method of dispensing a medication by a delivery device, said delivery device comprising:
a barrel having an internal cavity for receiving the medication, said internal cavity having an outlet connected to a delivery assembly for delivering the medication to a patient, said internal cavity having an inlet for introducing the medication into said internal cavity, a movable plunger in said internal cavity, and a drive assembly including a drive screw and actuator having internal threads for coupling to said drive screw for moving axially with respect to an axis of said drive screw, said plunger being movable from a first end toward a second end of said barrel by introducing the medication into said internal cavity;
wherein said method comprises
introducing the medication through said inlet of barrel to move said plunger from a first position toward said second end of said barrel and into contact with said actuator, and
rotating said drive screw to move said actuator and plunger toward said first end to dispense the medication through said outlet of said barrel to said delivery assembly.

18. The method of claim 17, said method further comprising orienting said plunger in said barrel at said first end, and thereafter introducing the medication into said barrel to move said plunger into contact with said actuator.

19. The method of claim 18, said method further comprising positioning said actuator at a predetermined location in said barrel corresponding to a predetermined volume in said barrel, and thereafter introducing the medication into said barrel to move said plunger into contact with said actuator.

20. The method of claim 18, further comprising rotating said drive screw to move said actuator in said barrel to a position corresponding to said predetermined volume before introducing said medication into said barrel.
